SEQUENCE LISTING (7 sequences)

SEQ ID NO: 1 - DNA, 119 bp, Homo sapiens
atatatttcttcatgaagacctcacagtaaaaataggtgattttggtctagctacagtgaaatctcgatggagtgggtcccatcagtttgaacagttgtctggatccattttgtggatg

SEQ ID NO: 2 - DNA, 2510 bp, Homo sapiens
cgcctcccggccccctccccgcccgacagcggccgctcgggccccggctctcggttataagatggcggcgctgagcggtggcggtggtggcggcgcggagccgggccaggctctgttcaacggggacatggagcccgaggccggcgccggccggcccgcggcctcttcggctgcggaccctgccattccggaggaggtgtggaatatcaaacaaatgattaagttgacacaggaacatatagaggccctattggacaaatttggtggggagcataatccaccatcaatatatctggaggcctatgaagaatacaccagcaagctagatgcactccaacaaagagaacaacagttattggaatctctggggaacggaactgatttttctgtttctagctctgcatcaatggataccgttacatcttcttcctcttctagcctttcagtgctaccttcatctctttcagtttttcaaaatcccacagatgtggcacggagcaaccccaagtcaccacaaaaacctatcgttagagtcttcctgcccaacaaacagaggacagtggtacctgcaaggtgtggagttacagtccgagacagtctaaagaaagcactgatgatgagaggtctaatcccagagtgctgtgctgtttacagaattcaggatggagagaagaaaccaattggttgggacactgatatttcctggcttactggagaagaattgcatgtggaagtgttggagaatgttccacttacaacacacaactttgtacgaaaaacgtttttcaccttagcattttgtgacttttgtcgaaagctgcttttccagggtttccgctgtcaaacatgtggttataaatttcaccagcgttgtagtacagaagttccactgatgtgtgttaattatgaccaacttgatttgctgtttgtctccaagttctttgaacaccacccaataccacaggaagaggcgtccttagcagagactgccctaacatctggatcatccccttccgcacccgcctcggactctattgggccccaaattctcaccagtccgtctccttcaaaatccattccaattccacagcccttccgaccagcagatgaagatcatcgaaatcaatttgggcaacgagaccgatcctcatcagctcccaatgtgcatataaacacaatagaacctgtcaatattgatgacttgattagagaccaaggatttcgtggtgatggaggatcaaccacaggtttgtctgctaccccccctgcctcattacctggctcactaactaacgtgaaagccttacagaaatctccaggacctcagcgagaaaggaagtcatcttcatcctcagaagacaggaatcgaatgaaaacacttggtagacgggactcgagtgatgattgggagattcctgatgggcagattacagtgggacaaagaattggatctggatcatttggaacagtctacaagggaaagtggcatggtgatgtggcagtgaaaatgttgaatgtgacagcacctacacctcagcagttacaagccttcaaaaatgaagtaggagtactcaggaaaacacgacatgtgaatatcctactcttcatgggctattccacaaagccacaactggctattgttacccagtggtgtgagggctccagcttgtatcaccatctccatatcattgagaccaaatttgagatgatcaaacttatagatattgcacgacagactgcacagggcatggattacttacacgccaagtcaatcatccacagagacctcaagagtaataatatatttcttcatgaagacctcacagtaaaaataggtgattttggtctagctacagtgaaatctcgatggagtgggtcccatcagtttgaacagttgtctggatccattttgtggatggcaccagaagtcatcagaatgcaagataaaaatccatacagctttcagtcagatgtatatgcatttgggattgttctgtatgaattgatgactggacagttaccttattcaaacatcaacaacagggaccagataatttttatggtgggacgaggatacctgtctccagatctcagtaaggtacggagtaactgtccaaaagccatgaagagattaatggcagagtgcctcaaaaagaaaagagatgagagaccactctttccccaaattctcgcctctattgagctgctggcccgctcattgccaaaaattcaccgcagtgcatcagaaccctccttgaatcgggctggtttccaaacagaggattttagtctatatgcttgtgcttctccaaaaacacccatccaggcagggggatatggtgcgtttcctgtccactgaaacaaatgagtgagagagttcaggagagtagcaacaaaaggaaaataaatgaacatatgtttgcttatatgttaaattgaataaaatactctctttttttttaaggtggaaaaaaaaaaaaaaaaaaaaaaaaaaaaaaaaaaaaaaaccc

SEQ ID NO: 3 - DNA, 20 bp, Artificial Sequence
  Description of Artificial Sequence primer
tagcctcaattcttaccatc

SEQ ID NO: 4 - DNA, 21 bp, Artificial Sequence
  Description of Artificial Sequence primer
cataatgcttgctctgatagg

SEQ ID NO: 5 - DNA, 13 bp, Artificial Sequence
  Description of Artificial Sequence reporter wt
catcgagatttca

SEQ ID NO: 6 - DNA, 13 bp, Artificial Sequence
  Description of Artificial Sequence reporter mut
catcgagatttca

SEQ ID NO: 7 - DNA, 31 bp, Artificial Sequence
  Description of Artificial Sequence stabilizer
ctgtagctagaccaaaatcacctatttttac

(19) United States
(12) Patent Application Publication
(10) Pub. No.: US 2007/0087350 A1
(43) Pub. Date: Apr. 19, 2007

(54) Use of a mutation in the braf gene for the determination of the malignancy of melanoma cells

(76) Inventor: Andreas Kappel, Konigstein (DE)

Correspondence Address: O'Melveny & Myers, 610 Newport Center Drive, 17th Floor, Newport Beach, CA, 92660-6429, US

(21) Appl. No.: 10/581,936

(22) PCT Filed: Nov. 26, 2004
(86) PCT No.: PCT/EP04/13398

§ 371 Date: Jun. 7, 2006

(30) Foreign Application Priority Data
Dec. 9, 2003 (EP) 03028074.7

(51) Int. Cl. C12Q 1/68

(52) U.S. Cl. 435/6

(57) ABSTRACT

The invention relates to the use of oligonucleotide probes comprising exon 15 of the BRAF gene or a part thereof comprising codon 599 for the detection of the malignancy of melanoma cells.

Use of a mutation in the BRAF gene for the determination of the malignancy of melanoma cells

The present invention relates to the use of oligonucleotide probes comprising a part of the BRAF gene for the determination of the malignancy of melanoma cells and a method for the determination of the malignancy of melanoma cells.

A fast determination of the malignancy of a tumor, that in particular corresponds with the occurrence of metastasising cells, is important to provide an optimized treatment of a tumor. To establish new methods for the diagnosis of the stage of an malignant melanoma tumor the identification of specific markers correlated with the malignancy of tumor cells is desired.

The BRAF gene coding for the BRAF kinase has been suggested to be implicated in tumorigenesis almost a decade ago (S. M. Storm & U. Rapp, Toxicol. Lett. 67, 201-210, 1993). However, no clinical data have been published that demonstrate a role for BRAF in human cancers.

In a recent publication (H. Davies et al., Nature 417, 949-954, 2002), the presence of a mutation in exon 15 of the BRAF gene, wherein the valine codon is replaced by a glutamic acid codon by a mutation in codon 599 (V599E) was described. The mutation leads to a constitutive active variant of the BRAF kinase. However, only a limited number of primary malignant melanoma was examined and the melanoma were not classified according to their clinical stage.

The availability of a large panel of markers may allow a better discrimination between different clinical stages of melanocytic tumors and a more refined treatment of the tumors in the future.

It is an objection of the present invention to provide a new marker correlated with the malignancy of a tumor and its use and to provide methods for the determination of the clinical stage of melanoma using said marker.

It was found that only some biopsy samples of human malignant melanoma harbour the V599E mutation in exon 15 of the BRAF gene at a high fraction. Surprisingly, the samples of patients bearing the V599E mutation derived from malignant, in particular metastasising tumors. Samples of patients encompassing only the wildtype (a valine codon at position 599) derived from less malignant in particular not metastasising primary tumors.

Based on the correlation of the V599E mutation with the malignancy of melanoma an embodiment of the present invention is the use of an oligonucleotide probe comprising exon 15 of the BRAF gene or a part thereof comprising codon 599 or the counterstrands thereto for the detection of the malignancy of melanoma tumors or cells. A particular exon 15 sequence of the BRAF gene is shown in Seq ID No. 1:

(Seq ID No. 1)

5′-AT ATA TTT CTT CAT GAA GAC CTC ACA GTA AAA ATA
GGT GAT TTT GGT CTA GCT ACA [symbol] AAA TCT CGA TGG
AGT GGG TCC CAT CAG TTT GAA CAG TTG TCT GGA TCC
ATT TTG TGG ATG-3′

The underlined nucleotides indicates the valine codon 599 of the wildtype BRAF gene (Seq. ID No. 2).

Oligonucleotides comprising a sequence according to Seq ID No. 1 or oligonucleotides comprising a sequence complementary to Seq ID No. 1 or a part of said sequences comprising codon 599 or an allelic variant thereof are preferred for the detection of the malignancy of melanoma cells.

In particular suitable parts of Seq ID No. 1 encompassing codon 599 of the BRAF gene possesses preferably at least 10, better at least 20 nucleotides.

The invention is not limited to the use of oligonucleotides derived from Seq ID No. 1. In particular all allelic variants thereof, in particular oligonucleotides bearing a mutation in codon 599 of the BRAF gene are also encompassed. Allelic variants are defined oligonucleotides encompassed from melanoma probes, e.g. from patients, which hybridises with an oligonucleotide with Seq ID No. 1 or its counterstand under stringent conditions. Allelic variants have usually a sequence homology of more than 60%, in particular of more than 80% to Seq ID No. 1. Particularly preferred are oligonucleotides with a sequence wherein the homology is at least 90%.

In an highly preferred embodiment of the invention oligonucleotides are used wherein the codon 599 (GTG, GTA, GTC or GTT) is replaced through a codon GAG or GAA coding for glutamic acid or through a codon GGG, GGA, GGC or GGT coding for aspartic acid, but also oligonucleotides with other mutations in codon 599 can be used for the present invention.

The described oligonucleotide probes can be used for an allelic association with the malignancy of melanoma tumors. This opens the possibility to create methods for genotyping the melanoma of individuals. Therefore an other embodiment of the present invention is a method for the detection of the malignancy of melanoma tumors wherein the presence of a mutation in codon 599 in exon 15 of the BRAF gene or a part thereof comprising codon 599 is determined in melanoma probes comprising oligonucleotides encompassing parts of the BRAF gene bearing codon 599 or a complementary strand thereto.

A method for the preparation of a genomic sample probe comprising nucleic acid sequences from exon 15 of the BRAF gene is described in (H. Davies et al., Nature 417, 949-954, 2002). The obtained oligonucleotide probes can be amplified by PCR using the primers Seq. ID No. 3 and Seq. ID No. 4. But not only nucleic acid sequences derived from the genomic BRAF gene can be used in the present invention, also the corresponding mRNAs and cDNAs or parts thereof can be used for the determination of the malignancy of melanoma cells. Such genomic DNAs, mRNAs, cDNAs, its amplification products or parts thereof are explicitly encompassed of the term “oligonucleotide” respectively of the term “nucleic acid sequences”.

The detection of a mutation in codon 599 of exon 15 of the BRAF gene can be carried out with all known methods, e.g. by sequencing the isolated or amplified oligonucleotides, by northern respectively southern blotting or by hybridising the isolated or amplified nucleic acid sequences on a biochip with suitable reporter oligonucleotides with a complementary sequence to exon 15 of the BRAF gene or a part thereof.

Suitable reporter are e.g. oligonucleotides comprising a sequence Seq. ID No. 5 (wildtype reporter) and Seq. ID No. 6 (mutant reporter) or a substantially homologous sequence, in particular with an homology of at least 80%, particularly preferred of at least 90%. Preferred reporter oligonucleotides are labelled. Possible well known labels are dyes, eg. Cy™3 or Cy™5 (Amersham Pharmacia), fluorophores or a radioactive labelling of oligonucleotides. The use of suitable reporter oligonucleotides for the determination of the malignancy of melanoma cells or tumors and said reporter itself are an other embodiment of the present invention.

A preferred method for the detection of malignancy of melanoma cells or tumors is characterised by the parallel hybridisation of an labelled wildtype reporter and an mutant reporter marked with a different label under stringent conditions. The genotype of the examined sample (homozygote wildtype, heterozygote mutant/wildtype, homozygote mutant/mutant) can be determined through the intensity ratio of the signals derived from the hybridised reporters.

The term “hybridise under stringent conditions” means that two oligonucleotides are capable to hybridise with one another under standard hybridisation conditions as described in Sambrook, et al. Molecular Cloning: A laboratory manual (1989), Cold Spring Harbor Laboratory Press, New York, USA. For this purpose, it is possible to use common stringent hybridization conditions (e.g. 60° C, 0.1×SSC, 0.1% SDS), e.g.

The following examples show that codon 599 of exon 15 of the BRAF gene, in particular a mutation leading to the replacement of the valine codon into a glutamic acid codon, is a valuable prognostic marker in the diagnosis of the clinical stage of tumor cells of a patient.

### EXAMPLES

In the present examples the BRAF mutation status of malignant melanoma that were either classified as “primary tumors” or “metastasising tumors”. Tumor samples were classified by the University of Wuerzburg, and DNA samples were kindly provided by this institution.

The BRAF exon 15 sequence from each sample was amplified in a PCR reaction containing 77 μl aqua (bidest), 10 μl 10× reaction buffer (Promega), 8 μl 25 mM MgCl2, 2 μl 10 mM dNTPs, 1 μl Taq Polymerase (Promega), 0.6 μl 100 μM primer 5′-tagcctcaattcttaccatc-3′ (Seq. ID No. 3), 0.6 μl 100 μM primer 5′biotin-cataatgcttgctctgatagg-3′ (Seq. ID No. 4), and 1 μl genomic DNA. Amplification parameters were: 2 min at 95° C., 35 cycles (30 sec at 95° C., 20 sec at 55° C., 25 sec at 72° C.) and 5 min at 72° C. The PCR amplicons were subsequently purified and addressed to a NanoChip™ DNA microarray as described recently (H. A. Behrensdorf et al., Nucl. Acids Res., e64, 2002). Subsequently, the cartridge was incubated with 0.1 M NaOH for 5 min, rinsed with 5 ml 50 mM histidine, and rinsed with 500 μl high salt buffer (50 mM NaCl, 500 mM NaPO₄ pH 7.5).

Labelled oligonucleotides that are complementary to either the wildtype BRAF sequence (wt, 5′ Cy3- CAT CGA GAT TTC A -3′ (Cy™3 labelled Seq. ID No. 5)) or the BRAF V599E variant (mut, 5′ Cy5-CAT CGA GAT TTC T -3′ (Cy™5 labelled Seq ID No. 6)), and the stabilizer oligonucleotide 5′-CTG TAG CTA GAC CAA AAT CAC CTA TTT TTA C -3′ (Seq. ID No. 7) were subsequently mixed and diluted in high salt buffer to a final concentration of 1 μM of each reporter oligonucleotide and 2 μM of the stabilizer oligonucleotide . The mixture was subsequently incubated with the microarray surface for 5min.

After washing the microarray with 1 ml high salt buffer, the array was scanned for Cy™3 and Cy™5 fluorescence, respectively. Subsequently, the array was washed with 175 μl low salt buffer (50 mM NaPO₄) three times at 34° C., and scanned for Cy™3 and Cy™5 fluorescence, respectively. This washing and scanning step was repeated at 35° C., 36° C. and 37° C. A robust discrimination was usually observed at 35° C. or 36° C., respectively. This point was reached when the green/red ratio in a homozygous wildtype control sample was >5:1, and the green/red ratio in a homozygous mutant control sample was <1:5, with a green/red ratio in a heterozygote sample arbitrarily set to 1:1. Genotyping of the samples was then performed using the NanoChip™ Workstation's software. The results are shown in the following table 1:

The analysis revealed the presence of two groups within the patients that were examined, one having a wildtype genotype, the other with a predominant mutant genotype. Surprisingly a correlation between the genotype and the malignancy of the tumor was found. The “wildtype” group (wt/wt) suffered from non-metastasising melanoma (examples 1 to 12), whereas the “mutant” group (V599E/wt; V599E/V599E) suffered from metastasising tumors (examples 13 to 29).

TABLE 1

| | Cy ™ 3 | Cy ™ 5 | | |
|---|---|---|---|---|
| Sample | fluorescence | fluorescence | Ratio | |
| No. | (Red) | (Green) | (Red:Green) | Robe Designation |
| 1 | 18.7 | 862.44 | 1:46.12 | wt/wt |
| 2 | 1.3 | 245.75 | 1:189.04 | wt/wt |
| 3 | 2.65 | 417.99 | 1:157.73 | wt/wt |
| 4 | 18.5 | 1036.85 | 1:56.05 | wt/wt |
| 5 | 2.05 | 358.77 | 1:175.01 | wt/wt |
| 6 | 0 | 208.80 | Inf. | wt/wt |
| 7 | 0 | 176.75 | Inf. | wt/wt |
| 8 | 0.85 | 202.28 | 1:237.98 | wt/wt |
| 9 | 0 | 170.77 | Inf. | wt/wt |
| 10 | 0.28 | 231.08 | 1:840.30 | wt/wt |
| 11 | 0 | 124.04 | Inf. | wt/wt |
| 12 | 1.3 | 140.89 | 1:108.37 | wt/wt |
| 13 | 1036.85 | 1036.85 | 1:1 | V599E/wt |
| 14 | 92.7 | 175.12 | 1:1.89 | V599E/wt |
| 15 | 16.45 | 39.28 | 1:2.39 | —/— |
| 16 | 1036.85 | 1036.85 | 1:1‘0 | V599E/wt |
| 17 | 6.45 | 377.78 | 1:58.57 | wt/wt |
| 18 | 17.65 | 23.53 | 1:1.33 | V599E/wt |
| 19 | 6.15 | 286.50 | 1:46.59 | wt/wt |
| 20 | 3.95 | 144.69 | 1:36.63 | wt/wt |
| 21 | 237.35 | 231.08 | 1.03:1 | V599E/wt |
| 22 | 109.35 | 124.04 | 1:1.13 | V599E/wt |
| 23 | 8.65 | 717.37 | 1:82.93 | wt/wt |
| 24 | 27.25 | 0 | Inf. | V599E/V599E |
| 25 | 0 | 158.82 | Inf. | wt/wt |
| 26 | 20.65 | 0 | Inf. | V599E/V599E |
| 27 | 16.85 | 1036.85 | 1:61.53 | wt/wt |
| 28 | 151.85 | 295.74 | 1:1.95 | V599E/wt |
| 29 | 1036.85 | 107.74 | 9.62:1 | V599E/V599E |

1-10. (canceled)

11. A method for the detection of the malignancy of melanoma cells in a patient sample, comprising the steps of:
providing a patient sample containing a BRAF sequence containing codon 599 of exon 15;
providing a first oligonucleotide probe comprising a first label, wherein the oligonucleotide probe is complementary to a wildtype BRAF sequence containing codon 599;
providing a second oligonucleotide probe comprising a second label, wherein the second oligonucleotide probe is complementary to a mutant BRAF sequence containing a mutation at codon 599;
contacting the patient sample with the first and second oligonucleotide probes to hybridize at least one of the first and second oligonucleotide probes with the patient sample; and
detecting the at least one of the first and second oligonucleotide probes hybridized with the patient sample.

12. The method of claim 11, wherein the first oligonucleotide probe comprises a sequence selected from the group consisting of Seq. ID No. 5, a sequence complementary to Seq. ID No. 5, a sequence substantially homologous to Seq. ID No. 5, and a sequence complementary to a sequence substantially homologous to Seq. ID No. 5.

13. The method of claim 11, wherein the first oligonucleotide probe comprises a sequence selected from the group consisting of Seq. ID No. 1, a sequence complementary to Seq. ID No. 1, a sequence substantially homologous to Seq. ID No. 1, and a sequence complementary to a sequence substantially homologous to Seq. ID No. 1.

14. The method of claim 11, wherein the second oligonucleotide probe comprises a sequence selected from the group consisting of Seq. ID No. 6, a sequence complementary to Seq. ID No. 6, a sequence substantially homologous to Seq. ID No. 6, and a sequence complementary to a sequence substantially homologous to Seq. ID No. 6.

15. The method of claim 11, wherein codon 599 codes for valine.

16. The method of claim 15, wherein codon 599 is selected from the group consisting of GTG, GTA, GTC, and GTT.

17. The method of claim 11, wherein codon 599 that contains the mutation codes for an amino acid selected from the group consisting of glutamic acid and aspartic acid.

18. The method of claim 17, wherein codon 599 that contains the mutation is selected from the group consisting of GAG, GAA, GGG, GGA, GGC, and GGT.

19. A method for the detection of the malignancy of melanoma cells in a patient, comprising the steps of:
providing a patient sample containing a BRAF sequence containing codon 599 of exon 15;
sequencing the patient sample; and
determining the presence of a mutation at codon 599 of exon 15.

20. The method of claim 19, wherein codon 599 that contains the mutation codes for a glutamic acid or an aspartic acid.

21. The method of claim 19, further comprising the step of amplifying the patient sample before sequencing the patient sample.

22. A method for the detection of the malignancy of melanoma cells, comprising the steps of:
providing an oligonucleotide probe selected from the group consisting of exon 15 of the BRAF gene, a part of exon 15 of the BRAF gene comprising codon 599, and the counterstrands thereto;
hybridizing the oligonucleotide probe with a wildtype reporter comprising a sequence according to Seq. ID No. 5 and a first label;
hybridizing the oligonucleotide probe with a mutant reporter comprising a second label, wherein the mutant reporter is selected from the group consisting of a sequence according to Seq. ID No. 6, a sequence complementary to Seq. ID No. 5, a sequence complementary to Seq. ID No. 6, a sequence with a homology of over 80% to Seq. ID No. 6, a sequence with a homology of over 80% to a sequence complementary to Seq. ID No. 5, and a sequence with a homology of over 80% to a sequence complementary to Seq. ID No. 6; and
detecting a signal from the first label and a signal from the second label.

23. The method of claim 22, further comprising the step of determining a ratio of the signal from the first label and the signal from the second label.

24. The method of claim 22, wherein the oligonucleotide probe is a part of exon 15 of the BRAF gene.

25. The method of claim 24, wherein the part of exon 15 of the BRAF gene is selected from the group consisting of Seq. ID No. 1, an oligonucleotide comprising a sequence complementary to Seq. ID No. 1, a part of Seq. ID No. 1 comprising codon 599, and an allelic variant of Seq. ID No. 1.

26. The method of claim 22, wherein the oligonucleotide probe comprises codon 599, wherein codon 599 codes for Valine (Val, V).

27. The method of claim 22, wherein the oligonucleotide probe comprises a mutation at codon 599.

28. The method of claim 27, wherein the mutation codes for a glutamic acid (Glu, E) or an aspartic acid (Asp, D).

29. The method of claim 22, further comprising the step of detecting a mutation in codon 599 of the oligonucleotide probe.
